(19) United States
(12) Patent Application Publication
(10) Pub. No.: US 2015/0044179 A1
(43) Pub. Date: Feb. 12, 2015

(54) CELL PREPARATION INCLUDING FAT CELL

(71) Applicant: Masanori Saeki, Tamano-shi (JP)

(72) Inventor: Masanori Saeki, Tamano-shi (JP)

(21) Appl. No.: 14/380,703

(22) PCT Filed: Feb. 22, 2013
(86) PCT No.: PCT/JP2013/054483

§ 371 Date: Aug. 22, 2014

(30) Foreign Application Priority Data
Feb. 24, 2012 (JP) 2012-038323

(51) Int. Cl. A61K 35/12

(52) U.S. Cl. CPC A61K 35/35
USPC 424/93.7

(57) ABSTRACT

A main objective of the present invention is to provide a cell preparation useful for treatment of diseases in bone joints and repairing muscles which have been degenerated or damaged. The present invention provides a cell preparation for treatment of diseases in bone joints which comprises a fat cell, and a cell preparation for repairing muscles which comprises a fat cell and a mesodermal stem cell.

### TECHNICAL FIELD

The present invention relates to a cell preparation useful for alleviation of pain caused by a disease of an interosseous joint and for treatment of the disease. In addition, the present invention relates to a cell preparation useful for restoration of a degenerated or damaged muscle.

### BACKGROUND ART

As a means for increasing the volume of a soft tissue of human being, injection of hyaluronic acid, transplantation of a fat cell, and the like are known, and are frequently used in the field of cosmetic surgery, plastic surgery and the like such as removal of face wrinkle, excavation treatment and breast reconstruction. However, while treatment by injection of hyaluronic acid is easy, there is a problem that the injected hyaluronic acid is absorbed in about a half year, and that thus durability of the effect is poor. In addition, also as for transplantation of a fat cell, the fat cell sometimes necroses or is absorbed in a living body after transplantation. Thus, there is a disadvantage that reoperation may be required or scar may occur in some cases.

In order to eliminate these disadvantages, transplantation of an adipose stem cell was proposed, and a method for efficiently separating an adipose stem cell has been developed (for example, see Patent Documents 1 and 2). By transplanting an adipose stem cell, fixation ratio of the cell after transplantation is improved, and a certain effect is recognized in regeneration of a soft tissue. However, the adipose stem cell obtained by such a method may include impurities in some cases, and there has been a possibility that the impurities cause necrosis of a fat cell or a lump (calcification) after transplantation. A fat cell is generally obtained by liposuction, and the sucked adipose is a mixture including a wide variety of impurities in addition to a fat cell and an adipose stem cell. Impurities which should be removed from such a mixture include blood (especially erythrocytes), fatal/vital cells, senescent cells and the like. On the other hand, in Patent Document 3 is proposed a method of injecting a cell preparation (Condensed Rich Fat: CRF (registered trademark)) including a healthy concentrated fat cell and an adipose stem cell prepared by removing impurities which cause calcification or fat necrosis from a mixture collected by liposuction including a fat cell and an adipose stem cell by centrifugation using a syringe equipped with a weight filter. By such a method, fixation ratio of the cells is improved, and a problem such as a lump hardly occurs. Thus, such a method is becoming widely accepted.

As described above, a wide variety of techniques for separation and concentration of a fat cell and an adipose stem cell are developed, and a fat cell and an adipose stem cell which are safe and excellent in fixability are coming to be provided. However, transplantation of a fat cell and an adipose stem cell has been applied to regeneration of a soft tissue in most cases so far, and efficacy of these cells in regard to, for example, alleviation of arthralgia or regeneration of the tissue in a joint has not been sufficiently examined. In addition, it has not been known that a muscle is restored by administering a fat cell and an adipose stem cell to a damaged muscle.

### PRIOR ART DOCUMENTS

### Patent Documents

Patent Document 1: JP 2005-519883 W
Patent Document 2: JP 2007-509601 W
Patent Document 3: JP 2007-533396 W

### SUMMARY OF THE INVENTION

### Problems to be Solved by the Invention

A main object of the present invention is to provide a cell preparation useful for alleviation of pain caused by a disease of an interosseous joint and for treatment of the disease. In addition, another object of the present invention is to provide a cell preparation useful for restoration of a damaged muscle.

### Solutions to the Problems

The present inventor made earnest investigations for solving the above-mentioned problems and found that injection of a fat cell to a patient complaining about arthralgia early alleviates the symptom and that the effect is sustained. Furthermore, the present inventor also found that injection of a combination of a fat cell and an adipose stem cell results in efficient regeneration of a bone tissue or a cartilage tissue in the joint in addition to the above-mentioned alleviation of arthralgia. In addition, the present inventor found that a damaged muscle is restored by administering a cell preparation including a fat cell and a mesodermal stem cell to a site of muscle damage. As a result of studies further continued on the basis of these findings, the present invention was completed.

That is to say, the present invention provides a cell preparation, a method for alleviating pain in an interosseous joint, a method for regenerating a tissue of an interosseous joint, a method for restoring a muscle, and the like of the following aspects:

item 1. A cell preparation for treating a disease of an interosseous joint, including a fat cell.
item 2. The cell preparation according to item 1, further including a mesodermal stem cell.
item 3. The cell preparation according to item 1, wherein the mesodermal stem cell is an adipose stem cell.
item 4. The cell preparation according to item 1, including 1 to 10 mesodermal stem cells per one fat cell.
item 5. The cell preparation according to item 1, wherein the fat cell is a cell having been subjected to gamma irradiation.
item 6. The cell preparation according to item 1, wherein the disease of an interosseous joint is any one kind selected from the group consisting of spinal disease, joint disease, autoimmune disease and osteonecrosis.
item 7. Use of a cell preparation including a fat cell for production of a therapeutic agent for a disease of an interosseous joint.
item 8. A cell preparation for use in the treatment of a disease of an interosseous joint, including a fat cell.
item 9. A method for alleviating pain in an interosseous joint, including a step of administering a cell preparation including a fat cell to the interosseous joint of a patient having a disease of the interosseous joint.
item 10. A method for regenerating an interosseous joint, including a step of administering a cell preparation including a fat cell and a mesodermal stem cell to the interosseous joint of a patient having a disease of the interosseous joint.
item 11. A cell preparation for restoration of a muscle, including a fat cell and a mesodermal stem cell.
item 12. The cell preparation according to item 11, wherein the mesodermal stem cell is an adipose stem cell.
item 13. The cell preparation according to item 11, including 1 to 10 mesodermal stem cells per one fat cell.
item 14. The cell preparation according to item 11, wherein the fat cell is a cell having been subjected to gamma irradiation.
item 15. Use of a cell preparation including a fat cell and a mesodermal stem cell for production of a preparation for restoration of a muscle.
item 16. A cell preparation including a fat cell and a mesodermal stem cell used for restoration of a muscle.
item 17. A method for restoring a muscle, including a step of administering a cell preparation including a fat cell and a mesodermal stem cell to a site of muscle damage of a patient having muscle damage.

### Effects of the Invention

According to the cell preparation including a fat cell of the present invention, it is possible to improve sliding of a joint or the like by a fat cell, an ECM (extracellular matrix) existing around a fat cell or the like, thereby enabling early alleviation of arthralgia, and an excellent effect in alleviation of pain caused by a disease of an interosseous joint can be obtained. In addition, for example, by injecting the cell preparation of the present invention to an intervertebral disk or the like, the cell preparation serves as a cushion and exhibits an effect of making motion of a vertebral body good (improvement). Furthermore, the effect of alleviating arthralgia by the cell preparation of the present invention lasts for a long time.

In addition, according to the cell preparation including a fat cell and a mesodermal stem cell of the present invention, pain caused by a disease of an interosseous joint is alleviated by the fat cell, during which time regeneration of a tissue in the joint such as a bone tissue, a cartilage tissue or a soft tissue by the mesodermal stem cell is enhanced. In addition, by using a combination of a fat cell and a mesodermal stem cell, not only the above-mentioned pain can be alleviated, but also take percentage of the mesodermal stem cell and efficiency of regeneration of the tissue can be still more enhanced, since the mesodermal stem cell is retained in the affected area by the action of the fat cell. Thus, the cell preparation of the present invention including a fat cell and a mesodermal stem cell is further effective for treatment of a disease of an interosseous joint.

In addition, the cell preparation of the present invention including a fat cell and a mesodermal stem cell has an excellent restoration effect on a muscle damaged by degeneration of the muscle or excision of the muscle by surgery. In addition, according to the cell preparation, pain caused by muscle damage can be early alleviated, and thus the cell preparation also contributes to improvement of QOL (Quality of Life) of the patient.

Conventionally, when a method of restoring a tissue by culturing a mesodermal stem cell such as an adipose stem cell is employed, there has been a possibility of contamination in the course of culturing and the like, and an infectious disease could have been caused after transplantation. However, since the fat cell and the mesodermal stem cell included in the cell preparation of the present invention can be directly separated and concentrated from the sucked adipose or the like without undergoing a step of cell culturing, the fat cell and the mesodermal stem cell can be prepared without contacting with the open air. Therefore, there is no danger of an infectious disease or the like. In addition, since the cell preparation of the present invention is generally prepared using a fat cell and a mesodermal stem cell collected from a patient himself/herself who requires treatment of a disease of an interosseous joint or restoration of a muscle, the cell preparation has no problem of immunorejection and is highly safe.

### BRIEF DESCRIPTION OF THE DRAWINGS

FIG. 1(a) shows an X-ray photograph showing the left knee joint of subject A before administration of a cell preparation, and FIG. 1(b) shows an X-ray photograph showing the left knee joint of subject A about 1 month after administration of the cell preparation.

FIG. 2(a) shows an X-ray photograph showing the right and left knee joints of subject B before administration of a cell preparation, and FIG. 2(b) shows an X-ray photograph showing the right and left knee joints of subject B about 1 month after administration of the cell preparation.

FIG. 3 shows a representative example of MR images showing that regeneration of a bone tissue was enhanced and osteonecrosis was improved or disappeared by administering a cell preparation.

### EMBODIMENTS OF THE INVENTION

### 1. Cell Preparation

The present invention provides the cell preparation for treating a disease of an interosseous joint and the cell preparation for restoration of a muscle described below. The cell preparations will be described in detail herein below.

### (1-1) Cell Preparation for Treating a Disease of an Interosseous Joint

The present invention provides a cell preparation for treating a disease of an interosseous joint, characterized in that the preparation includes a fat cell.

A fat cell is a cell which has functions of synthesizing, storing and releasing fat and which forms an adipose tissue. In addition, since a fat cell has been completely differentiated, the cell does not undergo cell division. The fat cell is classified into a white fat cell and a brown fat cell. Either of the cells may be used in the present invention, or a mixture of both the cells may be used. In the present invention, the origin of the fat cell is not specifically limited, and, for example, a fat cell of a mammal, preferably a fat cell of a primate, further preferably a human fat cell is used. In the present invention, as a fat cell, a fat cell alone may be used, or a mixture of an extracellular matrix (ECM) existing around a fat cell and a fat cell may be used. Specifically, in the present invention, a collected fat cell may be used as it is, or a fat cell prepared by removing the extracellular matrix from the collected fat cell may be used.

A fat cell is collected from an adipose tissue. A method for collecting an adipose tissue is not specifically limited, and appropriately selected from conventionally known methods, including, for example, liposuction, lipectomy, and defatting. Collection by liposuction is preferable, since liposuction is relatively easy and minimally invasive. A method for obtaining a fat cell from the collected fat is not specifically limited, and is preferably the method of 2. Method for preparing a cell preparation described below.

In addition, the cell preparation for treating a disease of an interosseous joint of the present invention may further include a mesodermal stein cell. By including a mesodermal stem cell, regeneration of a tissue in the interosseous joint is enhanced, and the therapeutic effect by the cell preparation of the present invention is still more increased.

The mesodermal stem cell used for the cell preparation of the present invention mainly indicates an intermediate stem cell which is to differentiate to a cell belonging to a mesodermal tissue such as bone, cartilage, myocardium, and fat. The origin of the mesodermal stem cell is not specifically limited, and, for example, a mesodermal stem cell of a mammal, preferably a mesodermal stem cell of a primate, further preferably a human mesodermal stein cell is used.

As an example of a mesodermal stein cell, an adipose stem cell, a bone marrow stem cell, a hematopoietic stem cell and the like are specifically mentioned, and these stem cells can be obtained from each tissue depending on the kind of the cell in accordance with a conventionally known method.

In the present invention, as an example of a mesodermal stem cell, an adipose stem cell is preferably mentioned. An adipose stem cell can be collected from an adipose tissue. The adipose tissue used as the source of the adipose stem cell can also be collected by any of the methods such as liposuction, lipectomy and defatting as with the above-mentioned fat cell, and the method is preferably liposuction from the viewpoint of preventing outflow or damage of the adipose stem cell and the viewpoint of safer collection. In addition, a preferable example of a method for obtaining an adipose stem cell from the collected fat is the method described in 2. Method for preparing a cell preparation described below.

When the cell preparation of the present invention includes a mesodermal stem cell, it is preferable that the mesodermal stem cell in a state of a mixture with a fat cell be used. When the cell preparation of the present invention includes a mesodermal stem cell, the content of the mesodermal stem cell in the cell preparation is not specifically limited so long as a therapeutic effect on a disease of an interosseous joint can be obtained. While it can be thought that the higher the content of the mesodermal stem cell is, the more excellent therapeutic effect can be obtained, the content is, for example, at least 1, preferably 5 or more, more preferably 1 to 10, further preferably 5 to 10 mesodermal stem cells per one fat cell. In addition, in a case of a cell preparation prepared by the method (c) described below including the steps of (1c) and (2c), the number of the mesodermal stem cells included in 1 mL of the cell preparation is generally 100 thousand or more, preferably 150 thousand or more, further preferably 150 to 200 thousand.

In the cell preparation of the present invention, by using an autologous fat cell (and an autologous mesodermal stem cell) for the purpose of autologous transplantation, immunorejection can be avoided in addition to enablement of early improvement of the symptom. However, preparation of the cell preparation of the present invention using a fat cell (and a mesodermal stem cell) derived from another person is not limited by the above. When using a cell derived from another person, it is preferable to carry out treatment for removing antigenicity such as, for example, gamma irradiation for the fat cell (including the extracellular matrix (ECM) existing around the fat cell) collected from another person before transplantation. While the mesodermal stem cell is a cell generally having no immunogenicity, a mesodermal stem cell which has been subjected to the above-mentioned treatment may also be used, if needed.

As a preferable example of the cell preparation of the present invention, a cell preparation including a fat cell and an adipose stem cell can be mentioned. Generally, an adipose tissue includes a fat cell and an adipose stem cell. Thus, as the cell preparation including a fat cell and an adipose stem cell, a collected adipose tissue may be used as it is, or a cell preparation prepared by removing impurities (for example, an anesthetic solution injected upon collection of fat (so-called tumescent fluid), an aged fat cell, blood, and tissue fluid) from the collected adipose tissue, a cell preparation prepared by removing the impurities and a part of a fat cell from the collected adipose tissue, and the like may be used. Preferable examples of a method for preparing the cell preparation include methods (a) to (c) described below.

In addition, a conventionally known pharmaceutically acceptable carrier, excipient, antiphlogistic, analgesic, immunosuppressive or the like can be added to the cell preparation of the present invention, if needed. In addition, collagen, a fibroblast, a growth factor, a proliferator, a cytokine or the like may be added thereto so long as the effect of the present invention is not negated.

In addition, for example, another cell preparation of which action of regenerating a tissue is known such as PRP (Platelet Rich Plasma) may be used in combination with the cell preparation of the present invention. Furthermore, by adding an angiotensin II receptor antagonist such as losartan to PRP, the effect of regeneration of a cartilage tissue can be still more augmented. However, from the viewpoint of improving the efficiency of regeneration of a tissue, for example, it is preferable to remove VEGFs (vascular endothelial growth factors) from PRP before use in a case where regeneration of a cartilage tissue is expected, and it is preferable to remove BMPs (bone morphogenetic proteins) before use in a case where bone regeneration is expected.

The cell preparation for treating a disease of an interosseous joint of the present invention exhibits an excellent therapeutic effect on a disease of an interosseous joint. The interosseous joint indicates a connection part of bones, and encompasses a cartilaginous joint connected by a cartilage or a combination of a cartilage and a fibrous connective tissue, a synovial joint connected by a cartilage and a synovial membrane covering a joint capsule, and a fibrous joint connected by a fibrous connective tissue. In the present invention, the interosseous joint especially indicates a part having mobility connected by a cartilaginous joint and a synovial joint. As an example of the interosseous joint, a vertebral joint, a knee joint, a foot joint, a toe joint, a hip joint, a hand joint, an elbow joint, a shoulder joint, a finger joint, an acromioclavicular joint, a sacroiliac joint and the like can be specifically mentioned. In addition, for example, in a case of a knee joint, soft tissues such as a ligament and a meniscus are encompassed by the above-mentioned interosseous joint, and in a case of a vertebral joint, a disk (intervertebral disk) and the like are encompassed by the above-mentioned interosseous joint.

In addition, examples of the disease of an interosseous joint include hernia (prolapse), deformation, degeneration, and inflammation, and specifically include spinal diseases such as intervertebral disk displacement and spondylosis deformans; joint diseases such as osteoarthritis (including hip osteoarthritis and knee osteoarthritis) and chronic arthrosis; autoimmune diseases such as rheumatoid arthritis; and osteonecrosis such as idiopathic osteonecrosis of knee joint and osteonecrosis of femoral head. Preferably, intervertebral disk displacement, osteoarthritis, chronic arthrosis, idiopathic osteonecrosis of knee joint and osteonecrosis of femoral head can be mentioned.

The dose of the cell preparation of the present invention is not specifically limited so long as the dose can give a therapeutic effect on the disease of an interosseous joint, and can be appropriately set depending on the size of the part of administration, the site of the disease, the degree of the disease, and sex, physique and the like of the patient. For example, the dose is 100 thousand to 4 million cells, preferably 200 thousand to 2 million cells, further preferably 200 thousand to 1 million cells as converted into the number of the fat cells. For example, when a cell preparation obtained by the method (c) described below including the steps of (1c) and (2c) is used, the dose is 0.5 to 20 mL, preferably 1 to 10 mL, further preferably 1 to 5 mL. In addition, for example, regarding the knee joint, 1 to 20 mL, preferably 1 to 10 mL, further preferably 5 to 10 mL of the cell preparation can be administered. In addition, regarding the intervertebral disk, 0.5 to 20 mL, preferably 1 to 15 mL, further preferably 1 to 2 mL of the cell preparation can be administered.

Examples of the administration method include, but not limited to, a method of injection to the part of the disease of an interosseous joint (for example, joint space and intervertebral disk (intervertebral disc)) using a syringe, a cannula or the like. In addition, upon injection, a still more excellent therapeutic effect can be obtained by stimulating the portion of injection with the tip of a needle or a cannula. Examples of the method of stimulation include, but not specifically limited to, a method of stimulating the site of injection by turning a cannula of which tip is obliquely cut in all directions upon injecting the cell preparation.

When the cell preparation of the present invention is administered to patients having these diseases, the cell preparation may be administered after carrying out a treatment by a known method, if needed. For example, in a case of intervertebral disk displacement, the cell preparation of the present invention can be administered (injected), after removing the prolapsed intervertebral disk by a conventionally known method such as PLDD (Percutaneous Laser Disc Decompression). By the action of the fat cell included in the cell preparation of the present invention, pain caused by the disease of an interosseous joint (arthralgia) is alleviated, and, furthermore, when a mesodermal stem cell is included, a bone tissue, a cartilage tissue, a soft tissue or the like in the interosseous joint is regenerated by the action of the cell.

### (1-2) Cell Preparation for Restoration of a Muscle

The present invention provides a cell preparation capable of restoring a muscle in a site of muscle damage, characterized in that the cell preparation includes a fat cell and a mesodermal stem cell.

The fat cell and the mesodermal stem cell used in the cell preparation for restoration of a muscle of the present invention, and the content of both the cells in the cell preparation and the like are as described in the section of “(1-1) Cell preparation for treating a disease of an interosseous joint”. In addition, as a preferable aspect of the cell preparation for restoration of a muscle of the present invention, a cell preparation including a fat cell and an adipose stem cell, which is a mesodermal stem cell, can be mentioned. Examples of the method for obtaining an adipose stem cell include the method described in 2. Method for preparing a cell preparation described below.

In addition, also in the cell preparation for restoration of a muscle, a conventionally known pharmaceutically acceptable carrier, excipient, antiphlogistic, analgesic, immunosuppressive or the like can be added, if needed, in addition to the fat cell and the mesodermal stem cell. In addition, collagen, a fibroblast, a growth factor, a proliferator, a cytokine or the like may be added thereto so long as the effect of the present invention is not negated.

In addition, for example, another cell preparation of which action of regenerating a tissue is known such as PRP (Platelet Rich Plasma) may be used in combination with the cell preparation of the present invention so long as the effect of the present invention is not negated. However, from the viewpoint of still more improving the efficiency of regeneration of a muscle tissue, it is preferable to remove transforming growth factor β1 (TGF β1) before use.

The cell preparation for restoration of a muscle of the present invention exhibits an excellent effect of restoration of a muscle when applied to a site of muscle damage. Muscle damage includes a case in which a muscle has been excised by surgery, injury or the like, in addition to myodegeneration, myofibrosis, destruction or necrosis of a muscle fiber, amyotrophy, muscular paralysis, muscle weakness and the like. In addition, examples of the site or kind of muscle to which the cell preparation of the present invention is applied include, but not specifically limited to, skeletal muscles such as trunk muscles (including a muscle of the upper or lower limb and pectoralis major); smooth muscles such as bladder sphincter; and a myocardium. For example, by administering the cell preparation of the present invention to a site of damage of the muscle of the upper limb, pain due to muscle damage can be early alleviated and the muscle is restored, thereby expansion of ROM (Range of Motion) or improvement in motor function of the upper limb can be realized. In addition, by administering the cell preparation of the present invention to the bladder sphincter of a patient presenting urination disorder such as urinary incontinence or frequent urination due to muscle weakness, damage or the like of bladder sphincter, a problem of urinary incontinence, incontinence or the like can be safely and easily eliminated by increasing muscle strength of the bladder sphincter. In addition, the cell preparation of the present invention can also be applied to the site of muscle damage of a patient having a muscular disease such as myocardial infarction, ischemia-reperfusion injury, muscular dystrophy (for example, Duchenne's muscular dystrophy (DMD)) or myositis (myopathy).

In addition, muscle damage is often painful, but the pain can be early alleviated by administering the cell preparation for restoration of a muscle of the present invention to the site of muscle damage. Without expecting limitative interpretation of the present invention, it is thought that such effect of alleviating pain is due to the fat cell included in the cell preparation for restoration of a muscle.

The dose of the cell preparation of the present invention when used for the purpose of restoration of a muscle is not specifically limited so long as the dose can give an effect of restoration of a muscle, and can be appropriately set depending on the size of the part of administration, the site of the disease, the degree of the disease, and sex, physique and the like of the patient. For example, the dose per unit volume of 10 mL of damaged muscle is 100 thousand to 4 million cells, preferably 200 thousand to 2 million cells, further preferably 200 thousand to 1 million cells as converted into the number of the fat cells. In addition, when a cell preparation obtained by the method (c) described below including the steps of (1c) and (2c) is used, the dose per unit volume of 10 mL of damaged muscle is 0.5 to 20 mL, preferably 1 to 10 mL, further preferably 1 to 5 mL.

The administration method of the cell preparation of the present invention when used for the purpose of restoration of a muscle is not specifically limited, and a conventionally known administration method can be employed. For example, a method of injection to the site of muscle damage with a syringe, a cannula or the like can be mentioned.

### 2. Method for Preparing a Cell Preparation

In the present invention, a fat cell can be collected, and, if needed, separated and concentrated to prepare the cell preparation of the present invention. In addition, when preparing a cell preparation including a fat cell and a mesodermal stem cell, a fat cell collected as a mixture of these cells may be used, or each cell may be individually collected, and thereafter mixed with the other cell to give the cell preparation of the present invention. For example, when the mesodermal stem cell is an adipose stem cell, since the adipose stem cell can be simultaneously prepared from the same tissue as the fat cell and easily prepared, it is preferable to obtain a mixture of a fat cell and an adipose stem cell from an adipose tissue to give the cell preparation of the present invention. In addition, more easily, an adipose tissue including these cells may be used.

When preparing the cell preparation of the present invention from an adipose tissue, methods (a) to (c) described below can be mentioned as preferable methods. However, these methods do not limit preparation of the cell preparation of the present invention from a tissue other than fat, and the cell preparation of the present invention can also be prepared by appropriately setting conditions, considering the kind or properties of the cell according to the method described below, even when the cell preparation of the present invention is prepared from a tissue other than an adipose tissue (for example, blood, bone marrow aspirate, or muscle). Each of the methods (a) to (c) will be described in detail herein below.

### Method (a)

The method (a) for preparing the cell preparation of the present invention includes a step (1a) of removing a liquid fraction from the collected fat to give a cellular fraction.

Here, the collected fat indicates a mixture of a cellular fraction and a liquid fraction derived from an adipose tissue that is collected from an adipose tissue by liposuction, lipectomy, defatting or the like. The cellular fraction includes a fat cell, an adipose stem cell and impurities (hemocytes, fatal/vital cells, senescent cells and the like). In addition, the liquid fraction includes a tumescent fluid, a cellular tissue fluid or the like.

Separation of the liquid fraction from the cellular fraction can be easily carried out by precipitating the cellular fraction by leaving the collected fat standing still. Examples of the method for removing the liquid fraction include, but not specifically limited to, decantation and suction. In addition, centrifugation, filtration or the like can be carried out, if needed.

Conditions for centrifugation and filtration are not specifically limited so long as the fat cell and the adipose stem cell are not damaged and the impurities can be removed. Examples of the conditions include the conditions of at 700 to 2,500×g for 1 to 15 minutes, preferably the conditions of at 2,000 to 2,200×g for 5 to 10 minutes.

In addition, when filtration is carried out, for example, blood, tissue fluid, anesthetic administered to the part from which the fat has been collected (a tumescent fluid) and the like are removed by letting the collected fat through a mesh filter with pores having a size of 10 to 300 μm, preferably 10 to 150 μm, more preferably 10 to 100 μm, further preferably 15 to 20 μm to give a mixture of a fat cell and an adipose stem cell on the filter. In addition, a container equipped with such a mesh filter may be charged with the collected fat containing impurities, and the fat may be subjected to centrifugation.

Thus, an adipose tissue including a fat cell (including an extracellular matrix) and an adipose stem cell from which a liquid fraction has been removed can be obtained, and the adipose tissue can be used as the cell preparation of the present invention.

### Method (b)

The method (b) for preparing the cell preparation of the present invention is characterized in that the method includes the following steps:

(1b) removing a liquid fraction from the collected fat to give a cellular fraction; and
(2b) separating a fat cell and an adipose stem cell from at least a part of the cellular fraction obtained in step (1b).

Here, step (1b) is as described in the above-mentioned step (1a). In step (2b), when a cell preparation including a fat cell is prepared, the whole of the cellular fraction obtained in step (1b) can be subjected to the treatment of step (2b) and only the separated fat cell can be used. In addition, when a cell preparation including a fat cell and an adipose stem cell is prepared, a cell preparation including a fat cell and an adipose stem cell can be obtained by separating an adipose stem cell from at least a part of preferably half of the amount of the cellular fraction obtained in step (1b) and further adding the adipose stem cell to the cellular fraction obtained in step (1b).

Here, examples of the method for separating an adipose stem cell include the method described in Patent Document 1. More specifically, in order to facilitate separation of a fat cell and an adipose stem cell by degrading intercellular junction, a protease is added to the cellular fraction. Examples of the protease include collagenase, trypsin, and lipase. One kind of the protease selected therefrom may be used alone, or two or more kinds of the proteases may be used in combination.

The method for separating a fat cell and an adipose stem cell from the cellular fraction treated with an enzyme is not specifically limited, and can be appropriately selected from the conventionally known methods. For example, the method may be centrifugation, filtration or the like. Conditions for centrifugation are not specifically limited so long as the fat cell and the adipose stem cell can be separated without damaging the cells. Examples of the conditions include the conditions of at 100 to 500×g for 1 to 20 minutes, preferably the conditions of at 100 to 300×g for 5 to 10 minutes. By subjecting the cellular fraction to such centrifugation, an adipose stem cell can be obtained as a precipitate, and a fat cell is concentrated in the supernatant. In addition, when filtration is carried out, an adipose stem cell can be obtained on a filter by letting the cellular fraction or a suspension thereof through a mesh filter with pores having a size of 10 to 100 μm, preferably 10 to 50 μM, further preferably 15 to 20 μm, and a fat cell can be obtained in the filtrate.

The fat cell and the adipose stem cell treated with an enzyme is preferably subjected to washing treatment after separation, and washing can be carried out with phosphate buffered saline (PBS), saline, Ringer's solution, dextran or the like. The washing treatment may be repeated multiple times if needed, or the cells may be subjected to centrifugation if needed, upon collecting the cells after washing. The conditions for centrifugation can follow the conditions employed upon separating a fat cell and an adipose stem cell from the cellular fraction treated with an enzyme.

After washing, the fat cell separated in the present step (2b) can be used as the cell preparation of the present invention. The cell preparation of the present invention obtained using the fat cell obtained in the present method (b) exhibits an excellent effect in alleviation of pain caused by a disease of an interosseous joint.

Furthermore, the present method (b) may include a step (3b) of mixing the cellular fraction obtained in step (1b) with the adipose stem cell obtained in step (2b) after step (2b). In the present step (3b), a cell preparation including a fat cell and an adipose stem cell can be obtained by mixing the cellular fraction obtained in step (1b) with the adipose stem cell obtained in step (2b). The cell preparation thus prepared includes an adipose stem cell in a high concentration, and not only is effective for alleviation of pain caused by a disease of an interosseous joint, but also exhibits an excellent effect on regeneration of a tissue of an interosseous joint. In addition, such a cell preparation including a fat cell and an adipose stem cell exhibits an excellent effect of restoration of a muscle on a damaged muscle, and simultaneously exhibits an excellent effect on alleviation of pain caused by muscle damage.

### Method (c)

The method (c) for preparing a cell preparation of the present invention includes the following steps of:

(1c) removing a liquid fraction from the collected fat to give a cellular fraction; and
(2c) removing impurities from the cellular fraction obtained in step (1c) to give a concentrate of a fat cell and an adipose stem cell.

Here, step (1c) is as described in step (1a). In step (2c), the cellular fraction is concentrated by removing impurities therefrom. The method for removing impurities and the method for concentration are not specifically limited, and can be appropriately selected from the conventionally known methods. As an easy method, for example, by using the syringe equipped with a weight filter described in Patent Document 3, impurities such as free oil released from the broken fat cell, fatal/vital cells and senescent cells are removed to give a concentrate of a healthy fat cell and an adipose stem cell. Such a syringe is commercially available, and a specific example thereof is a syringe manufactured for LIPOMAX-SC (manufactured by Medikan International Inc., Seoul, Korea).

More specifically, a syringe equipped with a filter therein is charged with the collected fat, and the fat is separated into a liquid fraction and a cellular fraction by centrifugation. Furthermore, free oil (drainage oil) is separated by the filter. The liquid fraction separated here is a liquid which has not been completely separated from the cellular fraction and mixed therewith in step (1c). That is to say, in the syringe, free oil, a concentrate of a fat cell and an adipose stem cell, and a liquid fraction are separated in the upper layer, the intermediate layer and the lower layer, respectively. By discarding the layers other than the intermediate layer, a concentrate of a healthy fat cell and an adipose stem cell can be obtained, and the concentrate can be used as the cell preparation of the present invention. Since a healthy fat cell and an adipose stem cell are selected and concentrated, the cell preparation thus obtained hardly causes necrosis, calcification or the like after transplantation, and is still more useful in alleviation and treatment of symptoms of a disease of an interosseous joint, restoration of a damaged muscle and alleviation of pain due to muscle damage.

Conditions for centrifugation for separating the fat into a liquid fraction and a cellular fraction are not specifically limited so long as the fat cell and the adipose stem cell are not damaged and the impurities are separated from the fat cell and the adipose stem cell. Examples of the conditions include the conditions of at 700 to 2,500×g for 1 to 15 minutes, preferably the conditions of at 2,000 to 2,200×g for 5 to 10 minutes.

In addition, the pore size of the filter is not specifically limited so long as a fat cell and an adipose stem cell cannot pass through the pore and impurities can pass through the pore. The size is, for example, 10 to 300 μm, preferably 10 to 150 μm, more preferably 10 to 100 μm, further preferably 15 to 20 μm.

In addition, the present method (c) may include a step (3c′) of separating a fat cell and an adipose stem cell from at least a part of the concentrate obtained in step (2c) and further mixing the obtained adipose stem cell with the concentrate obtained in step (2c). The method for separating an adipose stem cell is as described in step (2b) of the above-mentioned method (b). In the present step (3c′), when an adipose stem cell is separated using a protease, generally about 700 thousand or more, preferably about 1.5 million or more, further preferably about 2 million or more of an adipose stem cell per 1 cc of the concentrate obtained in step (2c) can be obtained. Since a healthy fat cell is selected and concentrated, and moreover an adipose stem cell is included in a higher concentration, the cell preparation prepared via the present step (3c′) exhibits a still more excellent effect on treatment of a disease of an interosseous joint and restoration of a damaged muscle.

In addition, the fat cell separated in the present step (3c′) can be washed, and thereafter used as the cell preparation of the present invention. Since a healthy fat cell is selected and concentrated, the fat cell obtained here exhibits a still more excellent effect in alleviation of a disease of an interosseous joint or pain caused by muscle damage.

In addition, the present method (c) may include a step (3c″) of micronizing the fat cell in the obtained concentrate after step (2c), and separating the micronized fat cell and an adipose stem cell as a mixture. Micronization of the fat cell can be carried out by a method generally employed in the art, and is not specifically limited. For example, a fat cell is cut with a drill or the like if needed, and thereafter a mixture of the micronized fat cell and an adipose stem cell is separated. As the method for separation, the above-mentioned conditions for centrifugation can be employed. Upon carrying out the present step (3c″), after step (2c), the concentrate may be subjected to micronization and centrifugation of a fat cell while the concentrate is remaining in the syringe. Since free oil is separated from the mixture of the micronized fat cell and an adipose stem cell after centrifugation, the free oil can be discarded to give a mixture of a micronized fat cell and an adipose stem cell.

By undergoing such a separation step, a cell preparation including a finer fat cell and an adipose stem cell in a high concentration can be prepared. Here, a fine fat cell indicates a fat cell having a size such that it can pass through a needle of which gauge is 26 to 30, preferably 18 to 30. In the present step (3c″), a treatment to micronize the fat cell can be carried out at least once, preferably once to several times, more preferably once to twice. By micronizing the fat cell, the concentration of the adipose stem cell per unit volume of the cell preparation can be increased, and the effect of alleviating a disease of an interosseous joint or pain derived from muscle damage, the effect of regeneration of a tissue inside an interosseous joint and the effect of restoration of a muscle can be exhibited still more remarkably.

In addition, a step (4c″) of separating an adipose stem cell from at least a part of the mixture of a micronized fat cell and an adipose stem cell obtained in step (3c″) and mixing the obtained adipose stem cell with the concentrate obtained in step (2c) may be included. The method for separating an adipose stem cell is as described in regard to step (2b) of the method (b).

The fat cell obtained by separating the adipose stem cell in the present step (4c″) can be washed, and thereafter used as a cell preparation including a fat cell of the present invention. Washing can be carried out by the method same as the above-mentioned washing treatment carried out for the adipose stem cell. Since a healthy, micronized fat cell is selected and concentrated, the fat cell obtained here exhibits a further remarkably excellent effect in alleviation of pain caused by a disease of an interosseous joint or muscle damage.

In the present method (c), by using the syringe described in Patent Document 3, all of the steps can be carried out without contacting the cells with the open air. For example, a cannula or the like may be attached to the syringe described in Patent Document 3, the syringe may be charged with the fat collected by liposuction, and the fat can be subjected to removal of impurities and a concentration treatment as it is to give a cell preparation. Thereafter, also upon administering the cell preparation, since the cell preparation can be directly transferred from the syringe to a syringe for injecting the cell preparation and administered to the affected part, a cell preparation having no problem such as contamination and high safety can be obtained. As an easy method, the present method (c) can be carried out using a commercially available kit, and a specific example of such a kit is LIPOMAX-SC (manufactured by Medikan International Inc.).

The cell preparation of the present invention can be obtained by any of the above-mentioned methods, and it is preferable that the cell preparation is a cell preparation including a micronized fat cell preferably prepared via the method (b) or (c), more preferably via the method (c), further preferably via step (3″) or (4″) of the method (c). Since impurities have been removed and a micronized, healthy fat cell is included in a high concentration, the cell preparation including the micronized fat cell obtained via step (4″) of the method (c) exhibits a remarkable effect on treatment of a disease of an interosseous joint (especially alleviation of pain). The cell preparation including a micronized fat cell and an adipose stem cell obtained via step (3″) of the method (c) includes an adipose stem cell in a high concentration, in addition to a micronized, healthy fat cell. Such a cell preparation can exhibit a further remarkably excellent effect on treatment of a disease of an interosseous joint and restoration of a muscle.

### 3. Method for Alleviating Pain Caused by a Disease of an Interosseous Joint

The present invention provides a method for alleviating pain in an interosseous joint, characterized in that the method includes a step of administering a cell preparation including a fat cell to the interosseous joint of a patient having a disease of the interosseous joint.

The method for collecting fat to be used in the method for alleviating pain of the present invention, the method for separating a fat cell, the method for preparing a cell preparation, the method for administration to a patient and the like are as described in the section of “1. Cell preparation” and “2. Method for preparing a cell preparation”. In the method for alleviating pain of the present invention, a therapeutically effective amount of cell preparation is administered to a patient in need of alleviation of pain in an interosseous joint. The dosage form is not specifically limited, and can be appropriately selected from conventionally known methods depending on the position and range in which alleviation of pain is expected, age of the patient, and the like.

### 4. Method for Regenerating a Tissue of an Interosseous Joint

The present invention provides a method for regenerating a tissue of an interosseous joint, characterized in that the method includes a step of administering a cell preparation including a fat cell and a mesodermal stem cell to the interosseous joint of a patient having a disease of the interosseous joint.

The method for collecting fat to be used in the method for regenerating a tissue of the present invention, the method for separating a mesodermal stem cell, the method for preparing a cell preparation, the method for administration to a patient and the like are as described in the section of “1. Cell preparation” and “2. Method for preparing a cell preparation”. In the method for regenerating a tissue of the present invention, a therapeutically effective amount of cell preparation is administered to a patient in need of regeneration of a tissue. The dosage form is not specifically limited, and appropriately selected from conventionally known methods depending on the position and range in which regeneration is expected, age of the patient, and the like.

### 5. Method for Restoring a Muscle

The present invention provides a method for restoring a muscle, characterized in that the method includes a step of administering a cell preparation including a fat cell and a mesodermal stem cell to a site of muscle damage of a patient having muscle damage.

The method for collecting fat to be used in the method for restoring a muscle of the present invention, the method for separating a mesodermal stem cell, the method for preparing a cell preparation, the method for administration to a patient and the like are as described in the section of “1. Cell preparation” and “2. Method for preparing a cell preparation”. In the method for restoring a muscle of the present invention, a therapeutically effective amount of cell preparation is administered to a patient in need of restoration of a muscle in a site of muscle damage. The dosage form is not specifically limited, and appropriately selected from conventionally known methods depending on the position and range in which restoration of a muscle is expected, age of the patient, and the like.

### 6. Others

The present invention provides use of a cell preparation including a fat cell for production of a therapeutic agent for a disease of an interosseous joint, and a cell preparation including a fat cell used for treatment of a disease of an interosseous joint. Furthermore, the present invention provides use of a cell preparation including a fat cell and a mesodermal stem cell for production of an agent for restoration of a muscle, and a cell preparation including a fat cell and a mesodermal stem cell used for restoration of a muscle. The fat cell, the mesodermal stem cell, the cell preparation, the disease of an interosseous joint, the muscle damage and the like are as described above.

### EXAMPLES

### Preparation of a Cell Preparation

For preparation of the cell preparation, LIPOMAX-SC (manufactured by Medikan International Inc., Seoul, Korea) kit was used. In the preparation step of the present cell preparation, it was possible to carry out the step without contacting the cells with the open air at all. The specific method is as follows.

(i) Using a syringe (a syringe manufactured for LIPOMAX-SC: manufactured by Medikan International Inc., Seoul, Korea), liposuction was carried out on each subject. A syringe containing the collected fat was left standing still at room temperature (about 25° C.) for about 10 minutes to separate the fat into a cellular fraction and a liquid fraction, and the liquid fraction containing a tumescent fluid was discarded. The remaining cellular fraction was used in Test Example 2 as an adipose tissue. Thereafter, the syringe was subjected to centrifugation at 2,200×g for 8 minutes. By centrifugation, the cellular fraction was separated into three layers of the upper layer (free oil), the intermediate layer (a fat cell and an adipose stem cell) and the lower layer (a liquid fraction such as a cellular tissue fluid). Thereafter, only the intermediate layer was left in the syringe, and the upper and lower layers were discarded, thereby a concentrate of a fat cell and an adipose stem cell was obtained. The gelling treatment was carried out for the concentrate to micronize a relatively large fat cell. The gelling treatment was carried out once to three times. A drill (Filler-Geller cutter: manufactured by Medikan International, Inc.) was used for gelling to cut the fat cell for micronization, and the fat cell was further centrifuged at 2,200×g for 5 minutes, thereby a mixture including a micronized fat cell and an adipose stem cell was obtained. The mixture thus obtained was used in Test Examples 2 to 5 described below as a cell preparation including a micronized fat cell and an adipose stem cell.

(ii) Next, extraction of an adipose stem cell was carried out. To the mixture of a fat cell and an adipose stem cell obtained in step (i), a 0.4% aqueous solution of a protease (trade name: Collagenase; manufactured by Worthington Biochemical Corp.) was added in an amount same as that of the mixture, and the mixture was mildly shaken. Thereafter, the mixture was incubated at 38° C. for 45 minutes.

(iii) After incubation, centrifugation was carried out at 200×g for 5 minutes, and the supernatant was collected. A fat cell was concentrated in the supernatant, and an adipose stem cell was concentrated in the precipitate.

(iv) Next, about 50 mL of dextran (manufactured by Otsuka Pharmaceutical Co., Ltd.) was added to 2 to 3 mL of the precipitate (a concentrate of an adipose stem cell) obtained in step (iii), and the mixture was mildly shaken. Thereafter, centrifugation was carried out at 200×g for 5 minutes, and the supernatant was discarded, thereby washing of an adipose stem cell was carried out. The procedure was repeated three times. The concentrate of an adipose stem cell obtained in the present step was used in the control group in Test Example 1 described below. In addition, the supernatant including a fat cell obtained in step (iii) was subjected to the same washing treatment, and thereafter used in Test Example 1 described below as a fat cell (and an extracellular matrix).

Furthermore, since the fat cell and the adipose stem cell derived from each patient were used, the above-mentioned procedures were carried out for each patient.

### Test Example 1

### Effect of Alleviating Pain Caused by Osteoarthritis (1)

The fat cell (including an extracellular matrix) obtained in the above-mentioned step (iv) was administered to subjects having knee osteoarthritis, and change in the symptoms was observed.

The subjects were 2 women (4 cases (both subjects had a disease in both the knee joints); average age: 68.5 years old). The fat used was collected from the lower abdomen of each subject. The amount of the collected fat was 102 mL and 95 mL, respectively. A fat cell (including an extracellular matrix) was obtained from the collected fat in accordance with the above-mentioned method for preparation of a cell preparation, and 7 mL of the fat cell was injected to each of the knee joints of each subject. In addition, as a control group, hyaluronic acid (Suvenyl (manufactured by CHUGAI PHARMACEUTICAL CO., LTD.: 2.5 mL) or 7 mL of the adipose stem cell obtained in the above-mentioned step (iv) (in both subjects, the site from which the fat was collected was the lower abdomen; the amount of the collected fat was 102 mL and 95 mL, respectively) was injected to the knee joint of another 2 subjects having knee osteoarthritis. In the present Test Example 1, upon carrying out injection to the knee joint, the procedure of stimulating the site of injection by turning the tip of a cannula in all directions was not carried out.

As a result, in the subject to whom a fat cell was injected, knee joint pain was reduced from the first day of the injection, and the effect lasted for about 1 month. In addition, as compared with the subjects to whom hyaluronic acid or an adipose stem cell was injected, the effect of alleviating knee joint pain was clearly developed from an earlier stage, and the effect of alleviating pain was also remarkable.

### Test Example 2

### Effect of Alleviating Pain Caused by Osteoarthritis (2)

The adipose tissue obtained in the above-mentioned step (i) was administered to subjects having knee osteoarthritis, and change in the symptoms was observed. The subjects were 3 women (6 cases (every subject had a disease in both the knee joints); average age: 69.7 years old). The fat used was collected from the lower abdomen of each subject. The amount of the collected fat was 95 mL, 100 mL and 105 mL, respectively. The adipose tissue was obtained from the collected fat in accordance with step (i) of the above-mentioned method for preparation of a cell preparation, and 7 mL of the adipose tissue was injected to each of the knee joints of each subject. In addition, as a control, hyaluronic acid (Suvenyl (manufactured by CHUGAI PHARMACEUTICAL CO., LTD.: 2.5 mL) was injected to the knee joint of another subject having knee osteoarthritis. In the present Test Example 2, upon carrying out injection to the knee joint, the procedure of stimulating the site of injection by turning the tip of a cannula in all directions was not carried out.

As a result, in any of the 3 subjects to whom an adipose tissue was injected, the effect of alleviating knee joint pain was developed earlier than in the subject to whom hyaluronic acid was injected, and the effect lasted for 1 month or more.

### Test Example 3

### Effect of Regeneration of a Tissue on Osteoarthritis

The cell preparation including a micronized fat cell and an adipose stem cell prepared in the above-mentioned step (i) (5 to 10 mL, including about 750 thousand to 2 million adipose stem cells) was injected to the knee joint of 9 women (14 cases; average age: 68.7 years old) having knee osteoarthritis, using a cannula of which tip was obliquely cut. The site of the disease, the specific site of liposuction, the amount of the collected fat, the amount of injection of the cell preparation, and presence or absence of the procedure of stimulating the site of injection by turning a cannula in all directions are shown in Table 1 below. Treatment for each subject, measurement results of the knee joint fissure and evaluation results on the basis of Kellgren and Lawrence classification which is an evaluation standard of an X-ray finding of knee osteoarthritis are summarized in Table 1 below.

TABLE 1

| | | | | | | | | | | | | | | | Subject |
|---|---|---|---|---|---|---|---|---|---|---|---|---|---|---|---|
| | | Subject A | Subject B | | Subject C | Subject D | | Subject E | | Subject F | Subject G | | Subject H | | I |
| Knee osteoarthritis | | Left | Right | Left | Right | Right | Left | Right | Left | Left | Right | Left | Right | Left | Left |
| Site from which | | Lower | Lower | | Lower | Femur | | Lower | | Lower | Lower | | Lower | | Femur |
| fat was collected | | abdomen | abdomen | | abdomen | | | abdomen | | abdomen | abdomen | | abdomen | | |
| Amount of collected fat | | 50 mL | 82 mL | | 90 mL | 80 mL | | 94 mL | | 76 mL | 76 mL | | 96 mL | | 102 mL |
| Presence or absence | | + | + | + | + | ++ | ++ | + | + | + | + | − | − | + | + |
| of procedure of | | | | | | | | | | | | | | | |
| stimulating site of | | | | | | | | | | | | | | | |
| injection by cannula | | | | | | | | | | | | | | | |
| Amount of injection of | | 5 mL | 7 mL | 7 mL | 8 mL | 7 mL | 8 mL | 6 mL | 7 mL | 7 mL | 8 mL | 6 mL | 6 mL | 8 mL | 9 mL |
| cell preparation | | | | | | | | | | | | | | | |
| Joint fissure | Before treatment | 2 | 0 | 0 | — | — | — | — | — | 1.6 | 0 | * | * | 2.4 | — |
| (mm) | After treatment | 3.3 | 2.6 | 2.6 | — | — | — | — | — | 3 | 1.8 | | | 4.4 | — |
| Kellgren and | Number of days | 3.5 weeks | 5 weeks | | 13 weeks | 12 weeks | | 7 weeks | | 11 weeks | 7.5 weeks | | 8 weeks | | 2 weeks |
| Lawrence | after treatment | | | | | | | | | | | | | | |
| classification | Before treatment | IV | IV | IV | II | III | IV | II | II | IV | III | II | III | IV | — |
| (Grade) | After treatment | II | II | II | I | II | III | I | I | II | II | I | II | III | — |

In the table, regarding knee osteoarthritis, left indicates that the subject has the disease in the left knee, and right indicates that the subject has the disease in the right knee.
In the table, regarding presence or absence of procedure of stimulating site of injection by cannula, + indicates presence of stimulation, ++ indicates presence of strong stimulation, and − indicates absence of stimulation.
In the table, regarding the size of joint fissure, — indicates that the size was not measured, and * indicates that the size remained almost unchanged after the treatment.
In the table, the number of days after treatment indicates the number of days elapsed from treatment until the Grade on the basis of Kellgren and Lawrence classification changed.
In the table, regarding Kellgren and Lawrence classification, — indicates that evaluation was not carried out since the number of days after treatment was small.

In addition, the specific evaluation items and evaluation standard of Kellgren and Lawrence classification are as follows.

TABLE 2

| | Narrowing of | Osteophyte | | Deformation of |
|---|---|---|---|---|
| Grade | joint fissure | formation | Consolidation | outline of joint |
| I | Doubted | Possible | None | None |
| II | Possible | Clear | None | None |
| III | Clear | Moderate | Moderate | Possible |
| IV | Moderate | Profound | Profound | Clear |

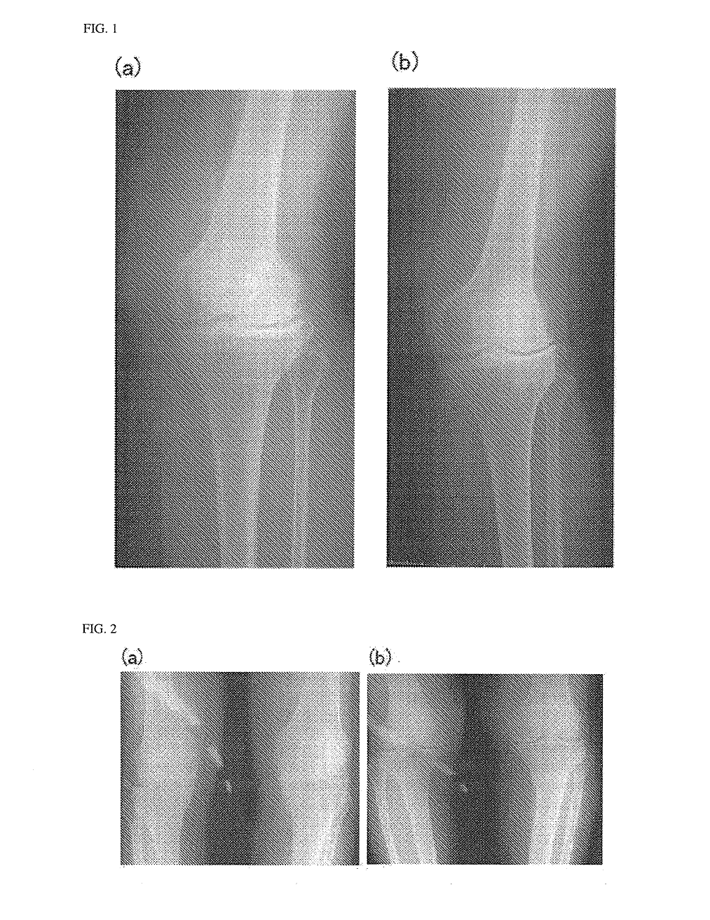

As representative exampled, X-ray photographs of the knee joints of subjects A and B are shown in FIGS. 1 and 2.

### Subject A

Age at the time of treatment: 72 years old, woman
Anamnesis: knee osteoarthritis (the left knee)

To the left knee joint was injected 5 mL of the cell preparation. The X-ray photographs of the joint fissure of the outside of the left knee joint of subject A taken before injection of the cell preparation and about 1 month after injection are shown in FIG. 1.

### Subject B

Age at the time of treatment: 60 years old, woman
Anamnesis: knee osteoarthritis (both right and left knees)

To the right and left knee joints was injected 7 mL each of the cell preparation. The X-ray photographs of the joint fissure of the outside of the right and left knee joints of subject B taken before injection of the cell preparation and about 1 month after injection are shown in FIG. 2.

### (Results)

Within 5 days after the day of treatment (average: 2 days), pain in the knee joint was remarkably improved in all of the subjects, and the state of improved pain lasted even after the above-mentioned period. In addition, 2 subjects who had been walking on cane became able to walk without a cane. Furthermore, 2 subjects who had been difficult to stand up became able to stand up, and walking remarkably improved in all of the subjects.

According to the X-ray photograph finding of the representative subjects A and B, improvement in knee osteoarthritis and expansion of joint fissure were shown. That is to say, as shown in FIG. 1, while the joint fissure of the left knee joint of subject A was 2.0 m before treatment (FIG. 1(a)), the joint fissure was expanded to 3.3 mm about 1 month after treatment (FIG. 1(b)). More specifically, while the grade of Kellgren and Lawrence classification was IV before treatment, the grade was improved to II about 1 month after treatment. In addition, as shown in FIG. 2, while joint fissure of the right and left knee joints of subject B was 0 mm before treatment (FIG. 2(a)), the joint fissure was expanded to 2.6 mm about 1 month after treatment (FIG. 2(b)). More specifically, while the grade of Kellgren and Lawrence classification was IV before treatment, the grade was improved to II about 1 month after treatment.

According to the results of the present Test Example 3, it was shown that regeneration of the tissue in the knee joint was enhanced by injecting the cell preparation including a micronized fat cell and an adipose stem cell to the knee joint of the subject having knee osteoarthritis. On the other hand, according to the X-ray photograph finding of the subjects to whom an adipose stem cell was injected as a control group of the above-mentioned Test Example 1, although regeneration of a tissue was found, speed of advance of regeneration of a tissue and improvement of the symptom were clearly slower than in subjects A and B.

### Test Example 4

### Effect of Regeneration of Tissue for Osteonecrosis

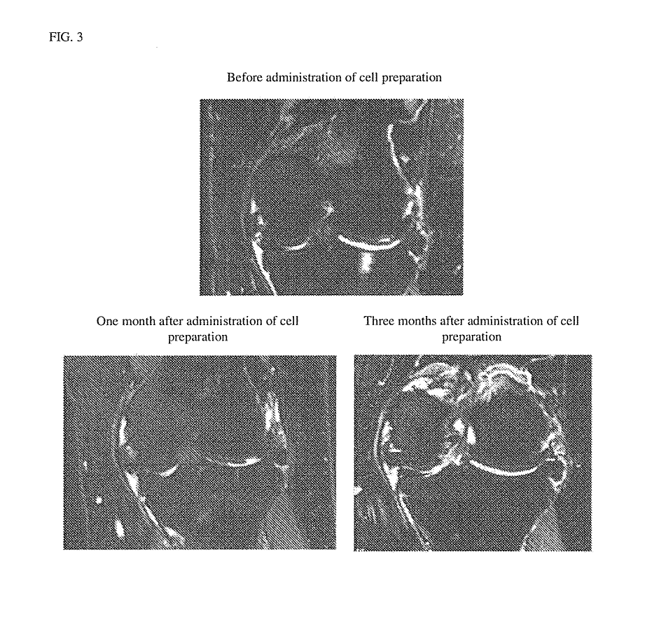

The cell preparation including a micronized fat cell and an adipose stem cell prepared in the above-mentioned step (i) (5 to 10 mL, including about 750 thousand to 2 million adipose stem cells) was injected to the site of osteonecrosis of 3 subjects of subjects A, B and J in Test Example 3 who additionally developed osteonecrosis in combination with knee osteoarthritis. Regarding subject J, the site of the disease, the specific site of liposuction, the amount of the collected fat and the amount of injection of the cell preparation are shown in Table 3 below. In addition, an MR image of the knee joint of subject A is shown in FIG. 3 as a representative example.

TABLE 3

| | | Subject J | |
|---|---|---|---|
| Knee osteoarthritis | | Right | Left |
| Site from which fat was collected | | Lower abdomen, | |
| | | both femurs | |
| Amount of collected fat | | 212 mL | |
| Presence or absence of procedure of | | + | + |
| stimulating site of injection by cannula | | | |
| Amount of injection of cell preparation | | 10 mL | 9 mL |
| Presence or absence of image of | | Present | Present |
| osteonecrosis before treatment | | | |
| Kellgren and | Number of days after | 12 weeks | 12 weeks |
| Lawrence | treatment | | |
| classification | Before treatment | IV | IV |
| (Grade) | After treatment | III | III |

In any of the subjects, symptoms such as walking difficulty and pain were remarkably improved within 2 days after injection of the cell preparation into the joint. In addition, regarding subject J, as shown in Table 3, while the grade of Kellgren and Lawrence classification was IV before treatment, the symptom was improved to III about 3 months after treatment. Furthermore, as shown in the MR image of the knee joint of subject A (FIG. 3), remarkable improvement in the image of osteonecrosis was found 1 month after injection of the cell preparation into the joint, and osteonecrosis was disappeared 3 months after the injection. When MR images in regard to the knee joints of subjects A and B were taken, improvement or disappearance of images of osteonecrosis and bone edema was found 1 to 3 months after injection of the cell preparation in either of the subjects.

### Test Example 5

### Effect of Restoration of a Muscle

The cell preparation including a micronized fat cell and an adipose stem cell prepared in the above-mentioned step (i) (10 to 50 mL, including about 4 million to 20 million adipose stem cells) was injected to the site of muscle damage of subjects having damage in quadriceps femoris muscle, damage in deltoid muscle of the upper arm or damage in pectoralis major (4 men; average age: 37.8 years old, 20 women; average age: 33.8 years old). For preparation of the cell preparation, fat collected from the lower abdomen or the femur of the subjects (the amount of collected fat: 50 to 200 mL) was used.

Regarding improvement of pain caused by muscle damage after injection of the cell preparation, the results were as follows: very little pain has been felt since injection (8 subjects: women); little pain has been felt since injection (4 subjects: men); pain disappeared 1 day after injection (10 subjects: women); and pain disappeared 2 to 3 days after injection (2 subjects: women). That is to say, it was shown that pain due to muscle damage is improved from an early stage by injection of a cell preparation including a fat cell and an adipose stem cell.

In addition, the range of motion (ROM: Range of Motion) of the joint of the upper limbs was expanded in all of the subjects within 3 weeks to 1 month after injection of the cell preparation, and improvement of motor function was found. That is to say, it was shown that a muscle is restored by administering the cell preparation to the site of muscle damage.

### 3. Conclusion

As described above, it was shown that pain caused by a disease of an interosseous joint can be early alleviated by injecting a fat cell to the affected part of a patient of knee osteoarthritis, and moreover that the effect lasts for a long time (Test Example 1). In addition, when an adipose tissue was injected, pain was early alleviated as in the case of Test Example 1, and the effect lasted for a still longer time (Test Example 2). Furthermore, by injecting a cell preparation including a micronized fat cell and a mesodermal stem cell (an adipose stem cell) in a high concentration to the affected part, regeneration of the tissue of the affected part was enhanced, in addition to the above-mentioned effect of alleviating pain (Test Example 3). Moreover, comparing the case where only an adipose stem cell was injected (control group of Test Example 1) with the case where the cell preparation including a micronized fat cell and an adipose stem cell was injected (Test Example 3), the effect of regeneration of a tissue was synergistically augmented when the cell preparation including a micronized fat cell and an adipose stem cell was injected. In addition, when the cell preparation including a micronized fat cell and an adipose stem cell was injected to the site of osteonecrosis (Test Example 4), regeneration of a bone tissue in the part of necrosis was found. Furthermore, it was shown that a muscle is restored by administering a fat cell and a mesodermal stem cell to the site of muscle damage (Test Example 5). It can be thought that, when a fat cell and a mesodermal stem cell such as an adipose stem cell are used in combination, the mesodermal stem cell is retained in the affected part by the action of the fat cell (including an extracellular matrix), thereby the efficiency of engraftment of a mesodermal stem cell and the efficiency of regeneration of a tissue are augmented.

According to the test examples described above, it was shown that the cell preparation of the present invention exhibits an excellent effect in treatment of a disease of an interosseous joint and restoration of a muscle.

1. A cell preparation for treating a disease of an interosseous joint, comprising a fat cell.

2. The cell preparation according to claim 1, further comprising a mesodermal stem cell.

3. The cell preparation according to claim 1, wherein the mesodermal stem cell is an adipose stem cell.

4. The cell preparation according to claim 1, comprising 1 to 10 mesodermal stem cells per one fat cell.

5. The cell preparation according to claim 1, wherein the fat cell is a cell having been subjected to gamma irradiation.

6. The cell preparation according to claim 1, wherein the disease of an interosseous joint is any one kind selected from the group consisting of spinal disease, joint disease, autoimmune disease and osteonecrosis.

7. Use of a cell preparation comprising a fat cell for production of a therapeutic agent for a disease of an interosseous joint.

8. A cell preparation for use in the treatment of a disease of an interosseous joint, comprising a fat cell.

9. A method for alleviating pain in an interosseous joint, comprising a step of administering a cell preparation comprising a fat cell to the interosseous joint of a patient having a disease of the interosseous joint.

10. A method for regenerating an interosseous joint, comprising a step of administering a cell preparation comprising a fat cell and a mesodermal stem cell to the interosseous joint of a patient having a disease of the interosseous joint.

11. A cell preparation for restoration of a muscle, comprising a fat cell and a mesodermal stem cell.

12. The cell preparation according to claim 11, wherein the mesodermal stem cell is an adipose stem cell.

13. The cell preparation according to claim 11, comprising 1 to 10 mesodermal stem cells per one fat cell.

14. The cell preparation according to claim 11, wherein the fat cell is a cell having been subjected to gamma irradiation.

15. Use of a cell preparation comprising a fat cell and a mesodermal stem cell for production of a preparation for restoration of a muscle.

16. A cell preparation comprising a fat cell and a mesodermal stem cell used for restoration of a muscle.

17. A method for restoring a muscle, comprising a step of administering a cell preparation comprising a fat cell and a mesodermal stem cell to a site of muscle damage of a patient having muscle damage.
